(19) United States
(12) Patent Application Publication
(10) Pub. No.: US 2018/0369219 A1
(43) Pub. Date: Dec. 27, 2018

(54) USING DABIGATRAN TO TREAT PANCREATITIS

(71) Applicant: Mayo Foundation for Medical Education and Research, Rochester, MN (US)

(72) Inventors: Baoan Ji, Ponte Vedra Beach, FL (US); Yan Bi, Ponte Vedra Beach, FL (US)

(21) Appl. No.: 16/013,849

(22) Filed: Jun. 20, 2018

Related U.S. Application Data
(60) Provisional application No. 62/524,957, filed on Jun. 26, 2017.

(51) Int. Cl. A61K 31/4439; A61P 1/18

(52) U.S. Cl. CPC A61K 31/4439; A61P 1/18

(57) ABSTRACT

This document provides materials and methods for treating pancreatitis (e.g., acute pancreatitis). For example, methods and materials for administering dabigatran to treat a mammal having pancreatitis (e.g., acute pancreatitis) under conditions wherein the severity of pancreatitis is reduced are provided. In some cases, methods and materials administering dabigatran to a mammal suspected of developing pancreatitis (e.g., acute pancreatitis) under conditions wherein the development of pancreatitis is slowed are provided.

### CROSS-REFERENCE TO RELATED APPLICATIONS

This application claims the benefit of U.S. Provisional Application Ser. No. 62/524,957, filed Jun. 26, 2017. The disclosure of the prior application is considered part of (and is incorporated by reference in) the disclosure of this application.

### BACKGROUND

### 1. Technical Field

This document relates to materials and methods for pancreatitis (e.g., acute pancreatitis). For example, this document provides methods for using dabigatran to treat a mammal having pancreatitis (e.g., acute pancreatitis).

### 2. Background Information

Acute pancreatitis (AP) is an inflammatory disease of the pancreas that causes significant morbidity and mortality. The incidence of first attack for AP in population studies over the past 2 decades from different countries ranges from 15 to 45 per 100,000 per year. In the United States, AP accounts for health care costs of $2.6 billion and for 275,000 hospital admissions each year. Hospital admissions due to pancreatitis have increased by at least 15% over the past 10 years. The mortality rate of AP ranges from 3% for patients with interstitial pancreatitis to 15% for patients with pancreatic necrosis. The risk of recurrence after the first attack of AP was noted to be ˜20% and in patients with at least one recurrence of AP, the potential for disease progression to chronic pancreatitis (CP) was estimated to be ˜35%. The prevalence of CP ranges from 13 to 52 per 100,000 populations. CP also is a risk factor for the development of pancreatic cancer—the 4th leading cause of cancer related death in the United States. The pooled relative risk estimate for pancreatic cancer among patients with CP is 13.3. In hereditary pancreatitis (HP), an inherited form of pancreatitis caused by a mutation in the PRSS1 gene, the pooled relative risk of developing pancreatic cancer can be as high as 69 with a lifetime risk of 40% to 55%. Unfortunately, despite more than 100 years of experience and thousands of experimental and clinical studies, understanding the development and progression of AP remains a challenge. There are still no targeted drugs available to treat the condition. Current treatment is limited to supportive care such as providing fluids, nutrition, and management of complications from the disease (Bi et al., Drug Therapy for Acute Pancreatitis. Current treatment options in gastroenterology, 13:354-68 (2015); and Stigliano et al., A review of the best evidence. Digestive and liver disease: official journal of the Italian Society of Gastroenterology and the Italian Association for the Study of the Liver 2017).

### SUMMARY

This document provides methods and materials for treating pancreatitis (e.g., acute pancreatitis). For example, this document provides methods for administering dabigatran to treat a mammal having pancreatitis (e.g., acute pancreatitis) under conditions wherein the severity of pancreatitis is reduced. In some cases, dabigatran can be administered to a mammal suspected of developing pancreatitis (e.g., acute pancreatitis) under conditions wherein the development of pancreatitis is slowed. As demonstrated herein, dabigatran can be used to treat pancreatitis within a mammal and/or can be used to slow or prevent the development of pancreatitis within a mammal.

In general, one aspect of this document features a method of treating a mammal having pancreatitis. The method comprises, or consists essentially of, administering a composition comprising dabigatran or a prodrug of the dabigatran to the mammal under conditions wherein the severity of the pancreatitis is reduced. The mammal can be a human. The pancreatitis can be acute pancreatitis, chronic pancreatitis, or hereditary pancreatitis. The composition can comprise dabigatran. The composition can comprise dabigatran as the sole active ingredient. The composition can comprise a prodrug of dabigatran. The composition can comprise a prodrug of dabigatran as the sole active ingredient. The prodrug can be dabigatran etexilate. The severity can be reduced by at least about 20 percent. The severity can be reduced by at least about 50 percent. The severity can be reduced by at least about 90 percent.

In another aspect, this document features a method of treating a mammal suspected of being at risk of developing pancreatitis. The method comprises, or consists essentially of, administering a composition comprising dabigatran or a prodrug of the dabigatran to the mammal under conditions wherein the development of the pancreatitis is slowed. The mammal can be a human. The pancreatitis can be acute pancreatitis, chronic pancreatitis, or hereditary pancreatitis. The composition can comprise dabigatran. The composition can comprise dabigatran as the sole active ingredient. The composition can comprise a prodrug of dabigatran. The composition can comprise a prodrug of dabigatran as the sole active ingredient. The prodrug of dabigatran can be dabigatran etexilate.

Unless otherwise defined, all technical and scientific terms used herein have the same meaning as commonly understood by one of ordinary skill in the art to which this disclosure pertains. Methods and materials are described herein for use in the present disclosure; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-D. The preventive effects of Camostat and Dabigatran on caerulein-induced acute pancreatitis. A. Timeline of caerulein (100 μg/kg×6), Camostat (300 mg/kg×2), and Dabigatran (200 mg/kg×2) administrations. B. Pancreas edema (pancreas/body weight ratio) measurement. C. Serum amylase test result of each group. D. Representative H&E sections obtained from mouse pancreas in each group. No Rx refers to no treatment.

FIGS. 2A-D. Prevention of hereditary pancreatitis by Ximelagatran, Camostat, and Dabigatran. A. Timeline of caerulein (100 μg/kg×6), Ximelagatran (40 mg/kg×2), Camostat (300 mg/kg×2), and Dabigatran (200 mg/kg×2) administrations. B. Pancreas edema (pancreas/body weight ratio) measurement. C. Serum amylase test result of each group. D. Representative H&E sections obtained from mouse pancreas in each group.

FIGS. 3A-C. Pancreatitis therapy by Camostat and Dabigatran in a model of hereditary pancreatitis. A. Timeline of caerulein (100 μg/kg×6), Camostat (300 mg/kg, Bid), and Dabigatran (200 mg/kg, Bid) administrations. B. Pancreas size (pancreas/body weight ratio) measurement. C. Representative H&E sections obtained from mouse pancreas in each group.

FIGS. 4A-D. Prevention of pancreatitis by Ximelagatran, Camostat, and Dabigatran in a mouse model expressing active trypsin. A. Timeline of caerulein (100 μg/kg×6), Ximelagatran (40 mg/kg×2), Camostat (300 mg/kg×2), and Dabigatran (200 mg/kg×2) administrations. B. Pancreas edema (pancreas/body weight ratio) measurement. C. Serum amylase test result of each group. D. Representative H&E sections obtained from mouse pancreas in each group.

### DETAILED DESCRIPTION

This document provides methods and materials for treating pancreatitis. For example, this document provides methods and materials for using dabigatran to treat a mammal having pancreatitis (e.g., acute pancreatitis). In some cases, this document provides methods for administering dabigatran to treat a mammal having pancreatitis (e.g., acute pancreatitis) under conditions wherein the severity of pancreatitis is reduced. In another example, this document provides methods and materials for using dabigatran to slow, reduce the likelihood of, or prevent the progression and/or development of pancreatitis (e.g., acute pancreatitis) within a mammal (e.g., a mammal suspected of being at risk of developing pancreatitis).

Any appropriate mammal (e.g., a human) can be treated as described herein. For example, a human having pancreatitis or suspected of being at risk of developing pancreatitis can be treated as described herein. Other examples of mammals that can be treated as described herein include, without limitation, non-human primates, monkeys, dogs, cats, horses, cows, pigs, sheep, rabbits, mice, and rats.

Any type of pancreatitis can be treated as described herein. For example, acute pancreatitis, hereditary pancreatitis, or chronic pancreatitis can be treated as described herein. In some cases, a mammal (e.g., a human) suspected to develop acute pancreatitis, hereditary pancreatitis, or chronic pancreatitis can be treated with dabigatran as described herein to slow, reduce the likelihood of, or prevent the progression and/or development of that pancreatitis.

Any appropriate method can be used to identify a mammal having pancreatitis or as being at risk for developing pancreatitis. For example, severe abdominal pain, elevated serum amylase, lipase exceeding three times the upper limit of a normal level, and/or abdominal imaging techniques (e.g., CT or MRI) that suggest pancreas parenchyma edema, necrosis, peri-pancreatic fat stranding, or pancreatic fluid collection can be used to identify a human or other mammal having pancreatitis. In some cases, a human's family health history can be evaluated to determine if the human is at risk of developing pancreatitis.

Once identified as having pancreatitis or as being at risk for developing pancreatitis, the mammal can be administered or instructed to self-administer dabigatran (e.g., a composition containing dabigatran). In some cases, a composition containing dabigatran administered to a mammal as described herein can include dabigatran as the sole active ingredient. For example, a mammal having pancreatitis or at risk for developing pancreatitis can be administered a composition containing dabigatran as the sole active ingredient. In some cases, a prodrug of dabigatran can be used in place of dabigatran or in addition to dabigatran. For example, a composition containing a prodrug of dabigatran can be administered to a mammal having pancreatitis or at risk for developing pancreatitis as described herein. As another example, a composition containing dabigatran and one or more different prodrugs of dabigatran (e.g., one, two, three, or more prodrugs of dabigatran) can be administered to a mammal having pancreatitis or at risk for developing pancreatitis as described herein. In some cases, a mammal having pancreatitis or at risk for developing pancreatitis can be administered a composition containing a prodrug of dabigatran as the sole active ingredient. An example of a prodrug of dabigatran includes, without limitation, dabigatran etexilate (e.g., dabigatran etexilate mesylate sold as PRADAXA®).

In some cases, dabigatran (or prodrugs of dabigatran) can be used to reduce the symptoms of pancreatitis. In some cases, dabigatran (or prodrugs of dabigatran) can be used to decrease pancreatic edema, pain, elevated serum amylase levels, inflammation in pancreas, pancreas atrophy and/or pancreas exocrine insufficiency.

In some cases, a composition containing dabigatran (and/or prodrugs of dabigatran) as described herein can be administered to a mammal having pancreatitis or at risk of developing pancreatitis as a combination therapy with one or more additional active agents to treat pancreatitis. In those cases where dabigatran (and/or prodrugs of dabigatran) are used in combination with one or more additional active agents to treat pancreatitis as described herein, the one or more additional active agents can be administered at the same time or independently. For example, a composition including dabigatran (and/or prodrugs of dabigatran) can be administered first, and the one or more additional active agents can be administered second, or vice versa.

In some cases, dabigatran (and/or prodrugs of dabigatran) can be formulated into a pharmaceutically acceptable composition for administration to a mammal having pancreatitis or at risk of developing pancreatitis. For example, a therapeutically effective amount of dabigatran (and/or prodrugs of dabigatran) can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules.

Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A pharmaceutical composition containing dabigatran (and/or prodrugs of dabigatran) can be designed for oral, parenteral (e.g., subcutaneous, intramuscular, intravenous, or intradermal administration), or inhaled administration. When being administered orally, a pharmaceutical composition containing dabigatran (and/or prodrugs of dabigatran) can be in the form of a pill, tablet, or capsule. Compositions suitable for parenteral administration include aqueous and non-aqueous sterile injection solutions that can contain anti-oxidants, buffers, bacteriostats, and solutes which render the formulation isotonic with the blood of the intended recipient; and aqueous and non-aqueous sterile suspensions which may include suspending agents and thickening agents. Compositions for inhalation can be delivered using, for example, an inhaler, a nebulizer, and/or a dry powder inhaler. The formulations can be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules, and tablets.

In some cases, a pharmaceutically acceptable composition including dabigatran (and/or prodrugs of dabigatran) can be administered locally or systemically. For example, a composition containing dabigatran (and/or prodrugs of dabigatran) can be administered systemically by an oral administration to or inhalation by a mammal (e.g., a human).

Effective doses can vary depending on the severity of the pancreatitis and/or risk of pancreatitis, the route of administration, the age and general health condition of the subject, excipient usage, the possibility of co-usage with other therapeutic treatments such as use of other agents, and the judgment of the treating physician.

An effective amount of a composition containing dabigatran (and/or prodrugs of dabigatran) can be any amount that reduces the severity of a symptom of a condition being treated (e.g., pancreatitis) without producing significant toxicity to the mammal. For example, an effective amount of dabigatran (or a prodrug of dabigatran such as dabigatran etexilate) can be from about 50 mg to about 300 mg (e.g., from about 50 mg to about 90 mg, from about 60 mg to about 130 mg, or from about 150 mg to about 220 mg). In one example, 75 mg dabigatran etexilate can be administered twice a day. In one example, 110 mg dabigatran etexilate can be administered twice a day. In another example, 150 mg dabigatran etexilate can be administered twice a day. The effective amount can remain constant or can be adjusted as a sliding scale or variable dose depending on the mammal's response to treatment. Various factors can influence the actual effective amount used for a particular application. For example, the frequency of administration, duration of treatment, use of multiple treatment agents, route of administration, and severity of the condition (e.g., pancreatitis) may require an increase or decrease in the actual effective amount administered.

The frequency of administration can be any frequency that reduces the severity of a symptom of a condition to be treated (e.g., pancreatitis) without producing significant toxicity to the mammal. For example, the frequency of administration can be from about once a week to about three times a day, from about twice a month to about six times a day, or from about twice a week to about once a day. The frequency of administration can remain constant or can be variable during the duration of treatment. A course of treatment with a composition containing dabigatran (or a prodrug of dabigatran such as dabigatran etexilate) can include rest periods. For example, a composition containing dabigatran (or a prodrug of dabigatran such as dabigatran etexilate) can be administered daily over a two-week period followed by a two-week rest period, and such a regimen can be repeated multiple times. As with the effective amount, various factors can influence the actual frequency of administration used for a particular application. For example, the effective amount, duration of treatment, use of multiple treatment agents, route of administration, and severity of the condition (e.g., pancreatitis) may require an increase or decrease in administration frequency.

An effective duration for administering a composition containing dabigatran (or a prodrug of dabigatran such as dabigatran etexilate) can be any duration that reduces the severity of a symptom of the condition to be treated (e.g., pancreatitis) without producing significant toxicity to the mammal. For example, the effective duration can vary from several days to several weeks, months, or years. In some cases, the effective duration for the treatment of pancreatitis can range in duration from about one week to about 10 years. Multiple factors can influence the actual effective duration used for a particular treatment. For example, an effective duration can vary with the frequency of administration, effective amount, use of multiple treatment agents, route of administration, and severity of the condition being treated.

In some cases, a course of treatment and the severity of one or more symptoms related to the condition being treated (e.g., pancreatitis) can be monitored. Any appropriate method can be used to determine whether or not the severity of a symptom is reduced. For example, the severity of a symptom of pancreatitis can be assessed using imaging techniques at different time points. In some cases, a scoring system can be used to assess the severity of acute pancreatitis based on clinical presentation (vital signs), lab work (serum creatinine, calcium, hemoconcentration, albumin, and/or liver tests), and imaging studies (e.g., imaging for severity of parenchymal and peri-parenchymal necrosis). For example, Ranson score of 3 or higher or an APACHE II (Acute Physiology and Chronic Health Evaluation II) score of 8 or higher can suggest severe acute pancreatitis. In the revised Atlanta criteria, severity of pancreatitis can be determined if there is associated organ failure (e.g., shock, pulmonary insufficiency, renal failure, gastrointestinal bleeding, disseminated intravascular coagulation, and/or severe metabolic disturbances) and/or local complications (e.g., pseudocyst, pancreatic necrosis, and/or pancreatic abscess).

The invention will be further described in the following examples, which do not limit the scope of the invention described in the claims.

### EXAMPLES

### Example 1—Treating Pancreatitis and/or Slowing Pancreatitis Progression

### Caerulein-Induced Pancreatitis

The secretagogue overstimulation model of experimental pancreatitis with caerulein, is a commonly used AP model (Zhan et al., Gastrointestinal Liver Physiology, 311:G343-55 (2016)). This model was accomplished by repeated intraperitoneal (ip) injections of caerulein (100 μg/kg/hour) once per hour (Huang et al., Gastroenterology, 144:202-10 (2013)).

### Animal Model of Hereditary Pancreatitis

A model of human hereditary pancreatitis (HP) was developed by using a bacterial artificial chromosome (BAC, clone RP11-701D14) harboring the full-length human PRSS1 gene containing an intact PRSS1 promoter, all exons and introns, and the 3′ untranslated region for faithful recapitulation of the gene's expression. An R122H point mutation was introduced by two rounds of genetic targeting using Galk-mediated recombineering (PRSS1^{R122H}) (Warming et al., Nucleic Acids Research, 33:e36 (2005)). This transgene expressed PRSS1^{R122H} at ˜50% of the endogenous human PRSS1 protein level as detected by Western blot (WB) and real-time RT-PCR. Taking into account that the PRSS1^{R122H} mutation was heterozygous, and that mutated PRSS1 constituted 50% of the total PRSS1 in human HP, this model does expressPRSS1^{R122H} at a physiologically relevant level. In this mouse model, spontaneous AP and CP were not observed. However, these mice developed more severe AP following the induction of AP with caerulein injection. More importantly, all of the PRSS1^{R122H} mice, but none of the WT mice, developed CP with an observed histopathology similar to that seen in human HP (Singhi et al., Am. J. Surg. Path., 38:346-53 (2014)). Therefore, this model faithfully recapitulates the histological changes observed during human HP (Singhi et al., Am. J. Surg. Path., 38:346-53 (2014)).

Animal Model with Active Trypsin Expression

A PACE-activated trypsinogen model in which the original enteropeptidase cleavage site (DDDDK) was replaced by a PACE cleavage site (RTKR), using site-specific mutagenesis of the wild type rat trypsinogen cDNA, was produced (Ji et al., J. Biol. Chem., 284:17488-98 (2009)). PACE, paired basic amino acid cleaving enzyme (also called furin), is a mammalian propeptide-processing endoprotease that is present in the trans-Golgi network of virtually all cells. To target expression of PACE-trypsinogen specifically to pancreatic acinar cells, conditional LGL (loxP-GFP-stop-loxP)-PACE-trypsinogen transgenic mice were bred with full-length, acinar specific, elastase promoter-regulated Cre transgenic mice. In these mice, an active form of trypsin was expressed only in the pancreatic acinar cells (Gaiser et al., Gut, 60:1379-88 (2011)).

### Administration of Therapeutic Drugs

Camostat mesilate (Santa Cruz), Dabigatran etexilate mesylate (a prodrug for Dabigatran, TRC Canada), and Ximelagatran (a prodrug for Melagatran, Sigma-Aldrich) were dissolved in 10% β-Cyclodextrin (Sigma-Aldrich). Each therapeutic agent was administered to mice by oral gavage twice a day at a dose of 300 mg/kg/day for dabagatran, 60 mg/kg/day for Ximelagatran, and 400 mg/kg/dat for camostat.

### Characterization of Pancreatitis

Pancreas edema, serum amylase, and histology were evaluated as described elsewhere (Gaiser et al., Gut, 60:1379-88 (2011); Huang et al., Gastroenterology, 144:202-10 (2013); and Daniluk et al., J. Clin. Invest., 122:1519-28 (2012)).

### Result

Pancreatitis Prevention with Camostat and Dabigatran in WT Mice

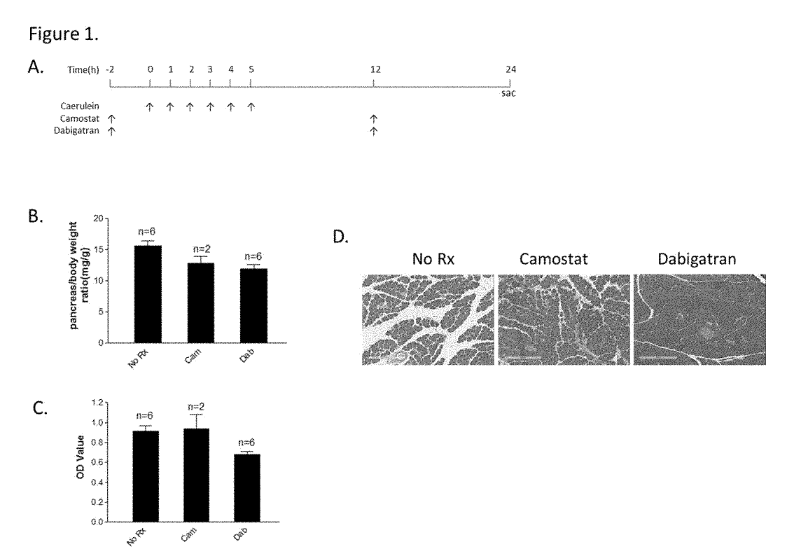

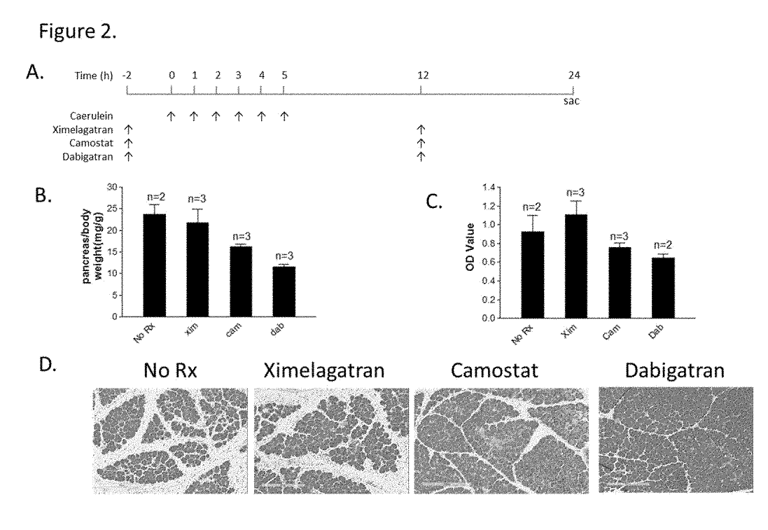

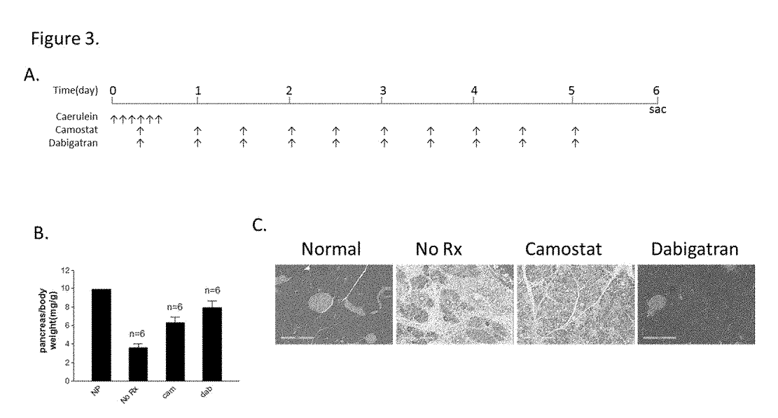

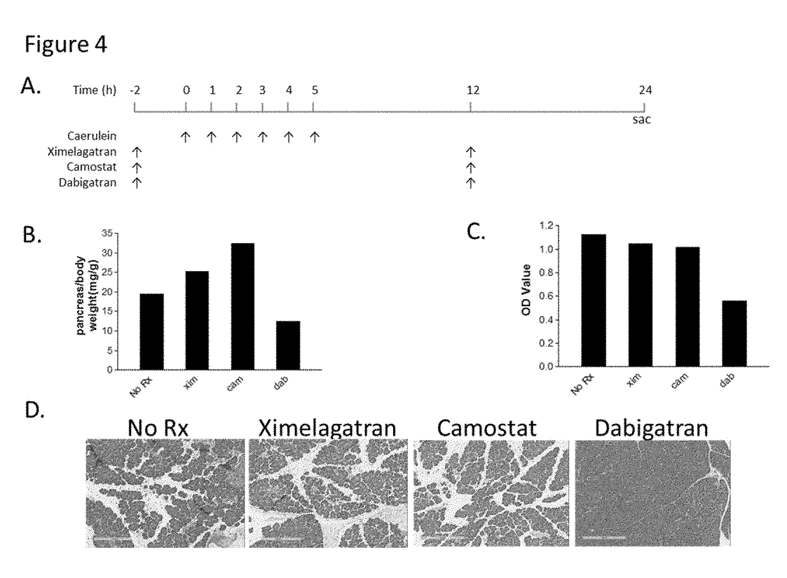

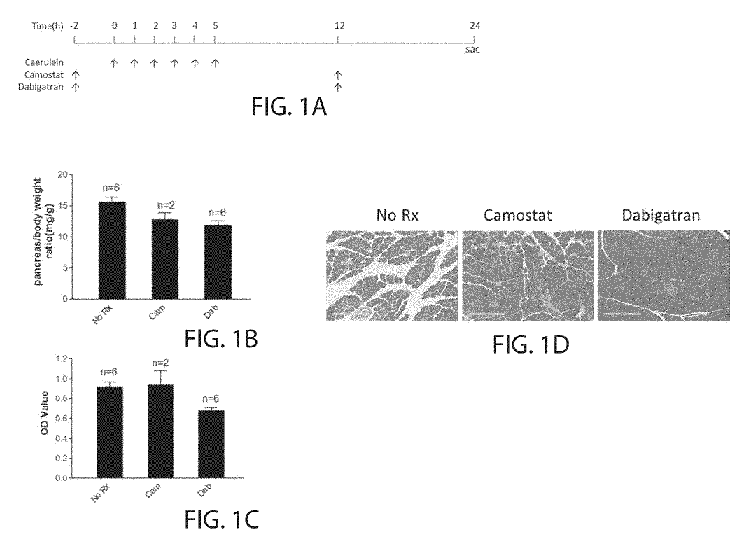

The effect of Dabigatran on experimental pancreatitis in WT mice was evaluated. Dabigatran and Camostat were administrated by oral gavage two hours before the initiation of AP by six hourly injections of caerulein (FIG. 1A). Twenty-four hours after the first dose of caerulein, the severity of pancreatitis was evaluated. Prophylactic administration of Dabigatran significantly reduced the pancreatic edema and serum amylase levels observed after caerulein injection (FIGS. 1B and 1C). Camostat was able to moderately reduce the level of pancreatic edema (FIG. 1B), but had no significant effect on serum amylase levels (FIG. 1C). Histological examination revealed that Dabigatran pretreatment dramatically reduced the severity of pancreatitis (FIG. 1D).

### Dabigatran Improved Pancreatic Health in the Mouse Model of Hereditary Pancreatitis

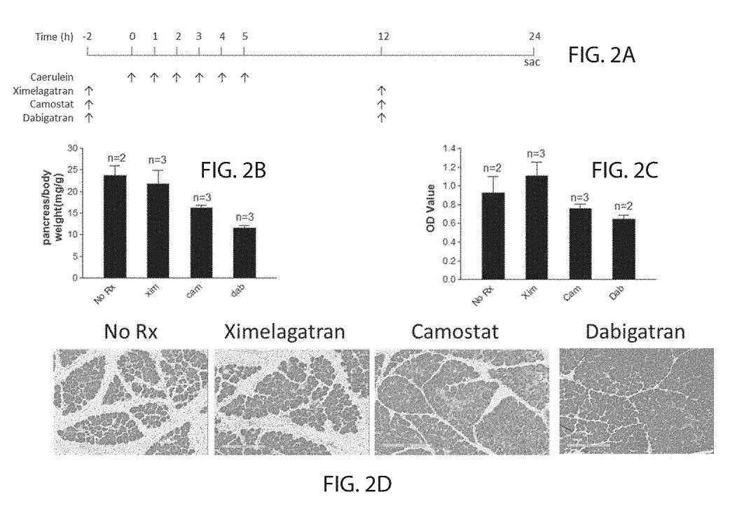

The preventive effect of Camostat and Dabigatran on the mouse model of human hereditary pancreatitis were tested. This model was developed by transgenic expression of the human PRSS1R122H gene using a bacteria artificial chromosome. This mouse model develops more severe AP than what is observed in the WT mice, supporting the role trypsin plays in the development and severity of pancreatitis. Mice that were pre-treated with Dabigatran, before induction of pancreatitis by caerulein injection, showed improved conditions including less pancreatic edema, decreased serum amylase levels, and more normal pancreatic histology (FIGS. 2A-2D). Pretreatment with Camostat only slightly improved these parameters (FIGS. 2A-2D). Interestingly, Ximelagatran, another thrombin inhibitor, showed no effects on reducing the severity of pancreatitis (FIGS. 2A-2D).

### Therapeutic Administration of Dabigatran Prevented the Development of Chronic Pancreatitis

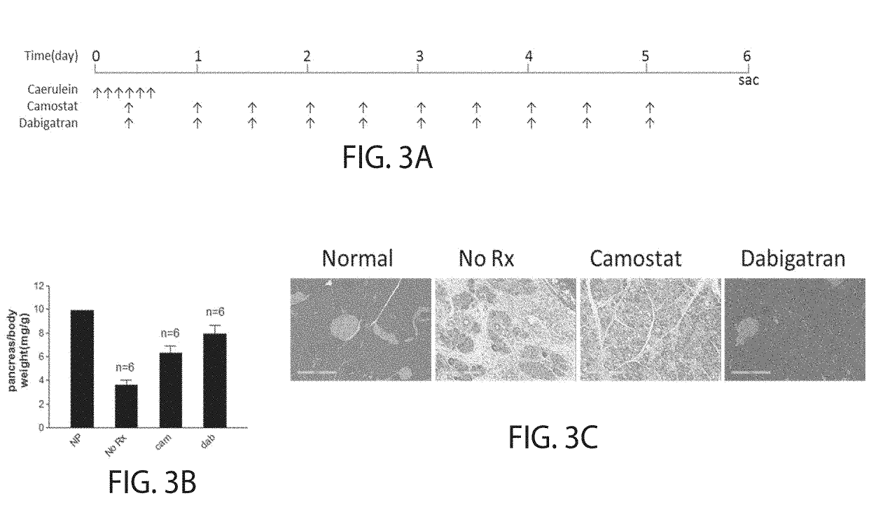

Although one episode of caerulein-induced AP in WT mice did not result in the progression to CP, transgenic expression of the human PRSS1R122H gene resulted in a more severe form of AP and led to the development of CP, with vast parenchymal cell loss. To investigate their potential therapeutic effects, Dabigatran and Camostat were administrated four hours after AP induction by caerulein injection, and the mice were treated for an additional five days (FIG. 3A). In mice that received no treatment, the pancreas became atrophic; identified by reduced weight (FIG. 3B), massive cell death, and increased inflammation (FIG. 3C). Treatment with Dabigatran effectively prevented the progression of AP to CP in this mouse model (FIGS. 3B and 3C). In contrast, Camostat treatment had no positive effects on preventing the development of CP (FIGS. 3B and 3C).

### Dabigatran Prevented the Development of Trypsin-Induced Acute Pancreatitis

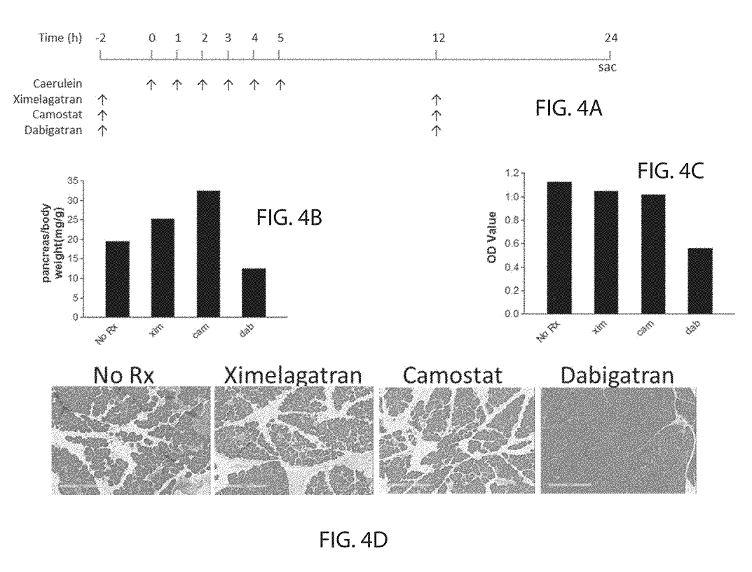

An animal model in which a mutant trypsinogen was expressed and activated only in pancreatic acinar cells was developed as described elsewhere (Gaiser et al., Gut, 60:1379-88 (2011)). These mice developed more severe AP, compared with WT mice. This information further emphasized the role trypsin plays in the development of pancreatitis. In this example, these mice were pretreated with Ximelagatran, Camostat, or Dabigatran two hours prior to the induction of AP by caerulein as well as seven hours following the final caerulein injection (FIG. 4A). Pretreatment with Dabigatran successfully prevented the development of severe AP in the mutant trypsinogen mice (FIGS. 4B and 4C). Examination of the histology revealed that pretreatment with Dabigatran resulted in tissue resembling normal WT animals (FIG. 4D). In contrast, pretreatment with Camostat or Ximelagatran had no ability to prevent the development of severe AP in the mutant mice as shown by increased pancreatic edema, significant change in serum amylase levels, acinar cell damage, and inflammatory cell infiltration (FIGS. 4A-4D).

These results demonstrate that Dabigatran can exert both preventive and therapeutic benefits in several experimental models of pancreatitis, including in a mouse model of human hereditary pancreatitis and that Dabigatran can be used to prevent pancreatitis and to treat pancreatitis.

### Example 2—Treating Pancreatitis

A human identified as having pancreatitis is administered 112 mg of dabigatran or 150 mg of dabigatran etexilate at least twice a day for one to three weeks. After this administration is initiated, a reduction in the severity of the pancreatitis is confirmed.

What is claimed is:

1. A method of treating a mammal having pancreatitis, wherein said method comprises administering a composition comprising dabigatran or a prodrug of said dabigatran to said mammal under conditions wherein the severity of said pancreatitis is reduced.

2. The method of claim 1, wherein said mammal is a human.

3. The method of claim 1, wherein said pancreatitis is acute pancreatitis, chronic pancreatitis, or hereditary pancreatitis.

4. The method of claim 1, wherein said composition comprises said dabigatran.

5. The method of claim 1, wherein said composition comprises said dabigatran as the sole active ingredient.

6. The method of claim 1, wherein said composition comprises said prodrug.

7. The method of claim 1, wherein said composition comprises said prodrug as the sole active ingredient.

8. The method of claim 1, wherein said prodrug is dabigatran etexilate.

9. The method of claim 1, wherein said severity is reduced by at least about 20 percent.

10. The method of claim 1, wherein said severity is reduced by at least about 50 percent.

11. The method of claim 1, wherein said severity is reduced by at least about 90 percent.

12. A method of treating a mammal suspected of being at risk of developing pancreatitis, wherein said method comprises administering a composition comprising dabigatran or a prodrug of said dabigatran to said mammal under conditions wherein the development of said pancreatitis is slowed.

13. The method of claim 12, wherein said mammal is a human.

14. The method of claim 12, wherein said pancreatitis is acute pancreatitis, chronic pancreatitis, or hereditary pancreatitis.

15. The method of claim 12, wherein said composition comprises said dabigatran.

16. The method of claim 12, wherein said composition comprises said dabigatran as the sole active ingredient.

17. The method of claim 12, wherein said composition comprises said prodrug.

18. The method of claim 12, wherein said composition comprises said prodrug as the sole active ingredient.

19. The method of claim 12, wherein said prodrug is dabigatran etexilate.
